(19) United States
(12) Patent Application Publication
(10) Pub. No.: US 2026/0221261 A1
(43) Pub. Date: Jul. 30, 2026

(54) METHOD FOR PROVIDING MACHINE-LEARNING-BASED DIET RECOMMENDATION SERVICE BY USING FOOD INTOLERANCE TEST (IGG ANTIBODY ANALYSIS), AND SERVICE PROVISION SERVER USED THEREFOR

(71) Applicant: BIOCOM CO.,LTD, Seoul (KR)

(72) Inventors: Tae Jun JEON, Seoul (KR); Hee Yong JUNG, Seoul (KR)

(73) Assignee: BIOCOM CO.,LTD, Seoul (KR)

(21) Appl. No.: 19/143,853

(22) PCT Filed: Oct. 26, 2023
(86) PCT No.: PCT/KR2023/016760

§ 371 Date: Aug. 4, 2025

(30) Foreign Application Priority Data
Dec. 26, 2022 (KR) 10-2022-0184148

(51) Int. Cl. G16H 20/60; G16H 10/20

(52) U.S. Cl. CPC G16H 20/60; G16H 10/20

(57) ABSTRACT

A method for providing a machine-learning-based diet recommendation service by using a food intolerance test (IgG antibody analysis), and a service provision server used therefor are disclosed. A service provision server generates food-specific-intolerance information of a user on the basis of the reaction value of an antigen-antibody reaction for each food antigen in the blood of the user; and generates customized diet information and recommended nutritional supplement information for the user on the basis of the food-specific-intolerance information of the user and the questionnaire answer information of the user. Since customized diet information for a user can be generated by reflecting the food-specific-intolerance information of the user, generated on the basis of the reaction value of an antigen-antibody reaction for each food antigen in the blood of the user, a diet from which risk due to a delayed allergy (IgG) is removed can be provided to the user.

### TECHNICAL FIELD

The present invention relates to a method for providing a machine-learning-based diet recommendation service by using a food intolerance test (IgG antibody analysis), and a service provision server used therefor, and more specifically, to a method for providing a machine-learning-based diet recommendation service by using a food intolerance test, and a service provision server used therefor, in which since customized diet information for a user may be generated by reflecting the food-specific-intolerance information of the user, generated on the basis of a reaction value of an antigen-antibody reaction for each food antigen in the blood of the user, a diet from which risk due to a delayed allergy (IgG) is removed may be provided to the user.

### BACKGROUND ART

A substance leading to food allergy is called a food antigen, and the food antigen results in an immune reaction in the body while being absorbed by the human body to cause characteristic clinical symptoms.

Food allergy symptoms to food antigens ingested in the body are mainly immediate reactions by specific IgE antibodies, but are continuously exposed to intestinal mucosa for up to seven days, thus causing a delayed reaction.

Since such delayed allergy (IgG) causes the delayed reaction for up to seven days, it is difficult to recognize the same as an allergy reaction, and it is also difficult to know the food that causes allergy, so there is a serious problem that the corresponding food is continuously ingested.

### DISCLOSURE

### Technical Problem

Accordingly, an object of the present invention may be to provide a method for providing a machine-learning-based diet recommendation service by using a food intolerance test, and a service provision server used therefor, in which since customized diet information for a user may be generated by reflecting the food-specific-intolerance information of the user, generated on the basis of a reaction value of an antigen-antibody reaction for each food antigen in the blood of the user, a diet from which risk due to a delayed allergy (IgG) is removed may be provided to the user.

The problem to be solved by the present invention is not limited to the above-mentioned problem, and may include other technical problems that may be clearly understood from the following description by those skilled in the art to which the present invention pertains.

### Technical Solution

To achieve the object described above, a method for providing a machine-learning-based diet recommendation service by using a food intolerance test according to the present invention may include: (a) generating, by a service provision server, food-specific-intolerance information of a user on the basis of a reaction value of an antigen-antibody reaction for each food antigen in the blood of the user; and (b) generating, by the service provision server, customized diet information and recommended nutritional supplement information for the user on the basis of the food-specific-intolerance information of the user and a questionnaire answer information of the user.

Preferably, the method may further include, after above (a) and before above (b), generating, by the service provision server, the predicted disease information of the user on the basis of the food-specific-intolerance information of the user and the questionnaire answer information of the user.

In addition, the method may further include (c) receiving, by the service provision server, ingested diet and nutritional supplement record information of the user from a terminal of the user.

Furthermore, the method may further include (d) generating, by the service provision server, an ingestion guidance message for the user on the basis of the ingested diet and nutritional supplement record information of the user.

Moreover, the method may further include (e) evaluating, by the service provision server, the propriety of customized diet information and recommended nutritional supplement information for the user on the basis of the health state information of the user, received from the terminal of the user.

Besides, the method may further include (f) updating, by the service provision server, the customized diet information and recommended nutritional supplement information for the user on the basis of the adequacy evaluation information.

Meanwhile, a service provision server according to the present invention may include: an operation part configured to generate food-specific-intolerance information of a user on the basis of a reaction value of an antigen-antibody reaction for each food antigen in the blood of the user, and generate customized diet information and recommended nutritional supplement information for the user on the basis of the food-specific-intolerance information of the user and the questionnaire answer information of the user.

Preferably, the operation part may generate the predicted disease information of the user on the basis of the food-specific-intolerance information of the user and the questionnaire answer information of the user.

In addition, the service provision server may further include a receiving part configured to receive ingested diet and nutritional supplement record information of the user from a terminal of the user.

Furthermore, the operation part may generate an ingestion guidance message for the user on the basis of the ingested diet and nutritional supplement record information of the user.

Moreover, the operation part may evaluate the propriety of customized diet information and recommended nutritional supplement information for the user on the basis of the health state information of the user, received from the terminal of the user.

Besides, the operation part may update and generate the customized diet information and recommended nutritional supplement information for the user on the basis of the adequacy evaluation information.

### Advantageous Effects

According to the present invention, since customized diet information for a user can be generated by reflecting the food-specific-intolerance information of the user, generated on the basis of the reaction value of an antigen-antibody reaction for each food antigen in the blood of the user, a diet from which risk due to a delayed allergy (IgG) is removed can be provided to the user.

The effects of the present invention are not limited to the above-mentioned effects, and include other effects that may be clearly understood from the following description by those skilled in the art to which the present invention pertains.

### DESCRIPTION OF DRAWINGS

FIG. 1 is a configuration view showing a service provision system for managing a customized diet on the basis of a delayed allergy test according to one embodiment of the present invention.

FIG. 2 is a functional block view showing a structure of a service provision server which executes a method for providing a machine-learning-based diet recommendation service by using a food intolerance test according to one embodiment of the present invention.

FIG. 3 is a signal flowchart showing a process of executing a method for providing a machine-learning-based diet recommendation service by using a food intolerance test according to one embodiment of the present invention.

### MODE FOR INVENTION

Hereinafter, the present invention will be described in more detail with reference to the drawings. It should be noted that the same components in the drawings are denoted by the same reference numerals wherever possible. In addition, detailed descriptions of well-known functions and configurations that may unnecessarily obscure the subject matter of the present invention will be omitted.

FIG. 1 is a configuration view showing a service provision system for managing a customized diet on the basis of a delayed allergy test according to one embodiment of the present invention. Referring to FIG. 1, the service provision system for managing the customized diet on the basis of the delayed allergy test according to one embodiment of the present invention may include a user terminal 100 and a service provision server 200.

The user terminal 100 may be a communication terminal such as a smart phone, etc., possessed by a user in order to use a customized diet management service on the basis of a delayed allergy test according to one embodiment of the present invention, and an application program required for using the service according to the present invention may be installed in such user terminal 100.

The service provision server 200 may be a server installed and operated by a business operator who provides a customized diet management service on the basis of a delayed allergy test according to one embodiment of the present invention, and the service provision server 200 may generate the food-specific-intolerance information of the user on the basis of a reaction value of an antigen-antibody reaction for each food antigen in the blood of the user, and generate customized diet information and recommended nutritional supplement information for the user on the basis of the food-specific-intolerance information of the user and the questionnaire answer information of the user.

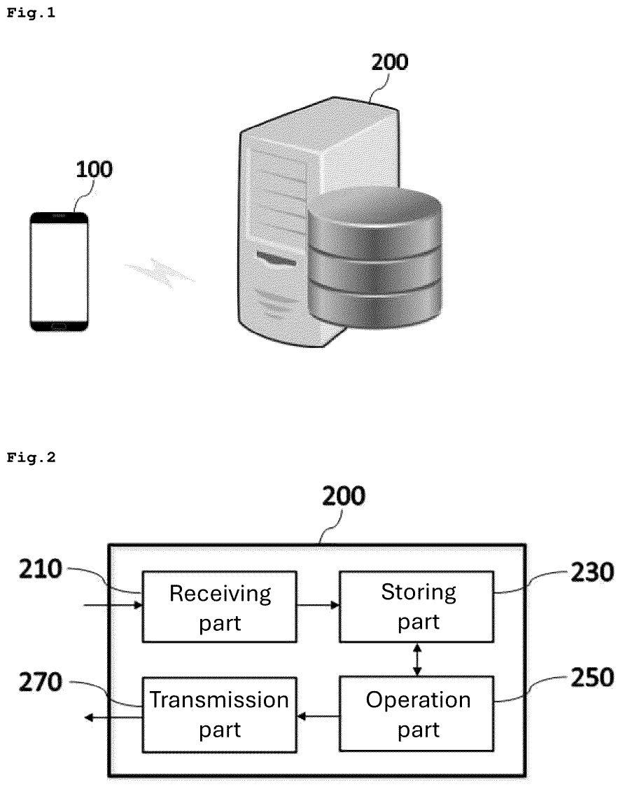

FIG. 2 is a functional block view showing a structure of a service provision server 200 which executes a method for providing a machine-learning-based diet recommendation service by using a food intolerance test according to one embodiment of the present invention. Referring to FIG. 2, the service provision server 200 which executes a method for providing a machine-learning-based diet recommendation service by using a food intolerance test according to one embodiment of the present invention may include a receiving part 210, a storing part 230, an operation part 250, and a transmission part 270.

The receiving part 210 of the service provision server 200 may receive questionnaire answer information of the user, ingested diet and nutritional supplement record information of the user, and health state information of the user from the terminal 100 of the user.

The operation part 250 of the service provision server 200 may generate food-specific-intolerance information of the user on the basis of the reaction value of the antigen-antibody reaction for each food antigen in the blood of the user, and generate customized diet information and recommended nutritional supplement information for the user on the basis of the food-specific-intolerance information of the user and the questionnaire answer information of the user.

The transmission part 270 of the service provision server 200 may send customized diet and recommended nutritional supplement information, and an ingestion guidance message for the user to the user terminal 100.

Meanwhile, a variety of received information in the receiving part 210, a variety of generated information in the operation part 250, and a variety of transmitted information in the transmission part 270 may be cumulatively stored in the storing part 230.

In addition, in implementing the present invention, a learning model for generating the customized diet information and recommended nutritional supplement information for the user may be stored in the storing part 230 of the service provision server 200.

The learning model in the present invention may be configured as an algorithm which generates the food-specific-intolerance information of a user on the basis of a reaction value of an antigen-antibody reaction for each food antigen of the user in the operation part 250 as described below, and generates the customized diet information and recommended nutritional supplement information for the user on the basis of at least one of food-specific-intolerance information, questionnaire answer information of the user, ingested diet information of the user, and health state information of the user, and may be formed as a neural network of various structures.

Specifically, such learning model may be machine-learned based on information, which is cumulatively stored in the storing part 230, such as the food-specific-intolerance information for each of a plurality of users, the questionnaire answer information of the user, the ingested diet information of the user, the health state information of the user, and the customized diet information and recommended nutritional supplement information, which are generated on the basis thereof.

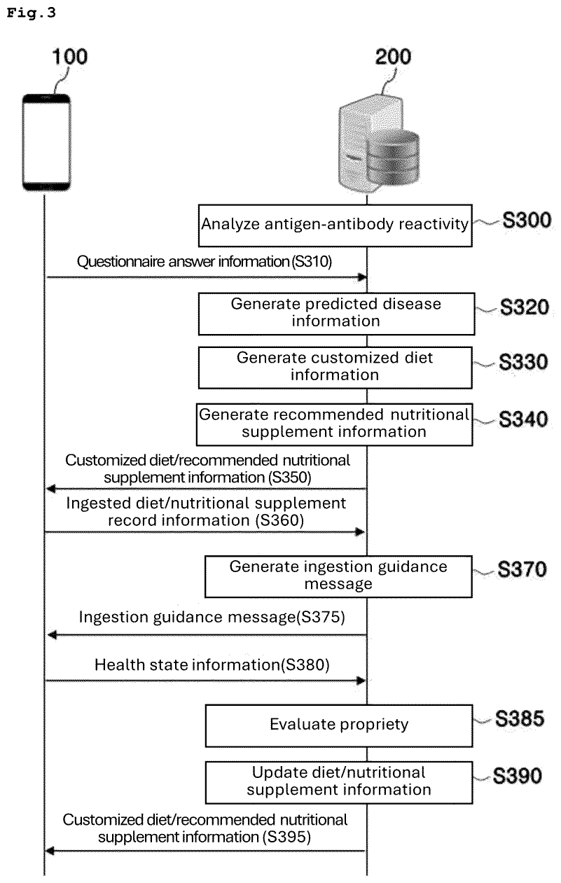

FIG. 3 is a signal flowchart showing a process of executing a method for providing a machine-learning-based diet recommendation service by using a food intolerance test according to one embodiment of the present invention. Hereinafter, referring to FIGS. 1 to 3, a process of executing a method for providing a machine-learning-based diet recommendation service by using a food intolerance test according to one embodiment of the present invention will be described later.

First of all, as a manager inputs information on the reaction value of the antigen-antibody reaction for each food antigen in the blood of the user, which has been collected in advance, into the service provision server 200, the operation part 200 of the service provision server 200 may analyze the reaction value for each food antigen, thereby generating intolerance information for each food of the user.

Specifically, in implementing the present invention, the information on the reaction value of the antigen-antibody reaction input in the service provision server 200 may become a reaction value for each of 90 types of food antigen as shown in Table 1, which corresponds to the diet of Koreans.

TABLE 1

| Classification | Food name |
|---|---|
| Meat/poultry | Beef, pork, chicken, lamb, duck, egg yolk, |
| (eight species) | egg white, quail egg |
| Seafood | Mackerel, flatfish, cod, anchovy, salmon, |
| (15 species) | eel, tuna, herring, octopus, squid, crab, |
| | oyster, lobster, shrimp, mussel |
| Dairy products | Cheese, goat's milk, yogurt, milk, milk |
| (five species) | protein (casein) |
| Grains | Gluten, rice, wheat, barley, buckwheat, |
| (eight species) | corn, oatmeal, rye |
| Fruit | Strawberry, lemon, mango, melon, banana, |
| (14 species) | pear, peach, apple, watermelon, orange, |
| | grapefruit, kiwi, pineapple, grape |
| Vegetables | Eggplant, potato, sweet potato, pepper, |
| (16 species) | carrot, radish, napa cabbage, mushroom, |
| | lettuce, spinach, cabbage, onion, |
| | cucumber, olive, tomato, pumpkin |
| Beans/nuts | Peanut, chestnut, almond, walnut, pea, |
| (11 species) | pine nut, sesame, pistachio, mung bean, |
| | soybean bean, sunflower seed |
| Spices/others | Mustard, cinnamon, oyster, green tea, |
| (11 species) | garlic, ginger, sugar, curry, coffee, |
| | cocoa, pepper |
| Additional fungi | Yeasts, Candida (fungus) |
| (two species) | |

More specifically, the operation part 250 of the service provision server 200 may divide a food-specific reactivity into five levels on the basis of the food-specific reaction values of the user and Table 2 below, and generate the classified level of the food-specific reactivity as intolerance information of the user for a corresponding food.

TABLE 2

| | Reaction value (R) Unit: ug/ml | | | | |
|---|---|---|---|---|---|
| | | 7.5 ≤ | 12.5 ≤ | 20.0 ≤ | |
| | R < 7.5 | R < 12.5 | R < 20.0 | R < 50 | R ≥ 50 |
| Reactivity | Level 1 | Level 2 | Level 3 | Level 4 | Level 5 |
| Intolerance | Level 1 | Level 2 | Level 3 | Level 4 | Level 5 |

Further, in implementing the present invention, the service provision server 200 may receive the questionnaire answer information of the user for questionnaire items as shown in Table 3 below from the user terminal 100 (S310).

TABLE 3

| No | Questionnaire Content |
|---|---|
| 1 | I enjoy drinking. [Y/N] (average number of drinks per week _{——} times) |
| 2 | I have a permit to smoke or a smoker in my family. [Y/N] |
| 3 | I am always tired even when I wake up. [Y/N] |
| 4 | I cannot sleep easily and I cannot sleep deeply. [Y/N] |
| 5 | I have a severe headache or migraine with an unknown cause. [Y/N] |
| 6 | I have frequent cramps in limbs and muscles. [Y/N] |
| 7 | I have depression or bipolar disorder. [Y/N] |
| 8 | Sometimes I am lethargic or have no strength. [Y/N] |
| 9 | I find it difficult to think deeply about something, and |
| | sometimes feel light-headed. [Y/N] |
| 10 | I have cold hands and feet and feel sensitive to cold. [Y/N] |
| 11 | I often catch a cold or have symptoms of decreased immunity. [Y/N] |
| 12 | I have various skin diseases such as atopy, acne and the like. [Y/N] |
| 13 | As a woman, I have menstrual problems or menopausal symptoms. [Y/N] |
| 14 | As a man, I have urination problems, sexual dysfunction, and prostate diseases. [Y/N] |
| 15 | I feel flatulent or have a symptom of abdominal fullness. [Y/N] |
| 16 | I have a symptom of gastresophageal reflux. [Y/N] |
| 17 | I suffer from constipation, diarrhea, or irritable bowel syndrome. [Y/N] |
| 18 | I have arthritis or osteoporosis. [Y/N] |
| 19 | I suffer from allergy, rhinitis, or asthma. [Y/N] |
| 20 | I have a symptom of hair loss or am under treatment. [Y/N] |
| 21 | I often eat instant food. [Y/N] |
| 22 | I am going on a diet or I am managing a diet. [Y/N] |
| 23 | I have a lack of concentration and have distracted or hyperactive behaviors. [Y/N] |
| 24 | I am taking an antihistamine-based drug. [Y/N] |

Accordingly, the operation part 250 of the service provision server 200 may generate the predicted disease information of the user on the basis of the questionnaire answer information of the user for the questionnaire items and the intolerance information of the user for each food (S320). For example, when the reaction information for the questionnaire content No. 2 is [Y] and the response information for the questionnaire content No. 21 is [Y], the operation part 250 of the service provision server 200 may primarily retrieve disease information which may occur in a smoker through the storing part 230, secondarily retrieve disease information which may occur due to instant food through the storing part 230, and generate disease information (for example, diabetes) commonly included in the primary and secondary retrieved results as the predicted disease information of the corresponding user.

In addition, the operation part 250 of the service provision server 200 may generate lactose intolerance as the predicted disease information of the user when the corresponding user's intolerance to the dairy products (five types) of above Table 1 is all equal to or higher than a predetermined reference level (for example, level 4).

Furthermore, the operation part 250 of the service provision server 200 may generate lactose intolerance as the predicted disease information of the user when the response information for the questionnaire content No. 15 or 17 is [Y] and the corresponding user's intolerance in the dairy products (five types) of above Table 17 is all equal to or higher than a predetermined reference level (for example, level 4).

Meanwhile, the operation part 250 of the service provision server 200 may generate customized diet information for the user through a machine-learning analysis by using a learning model on the basis of the predicted disease information of the user and/or intolerance information of the user for each food (S330).

For example, when the predicted disease information of the user is diabetes, the operation part 250 of the service provision server 200 may retrieve diet information for a diabetic patient previously stored in the storing part 230, thereby generating customized diet information for the user.

In addition, when the predicted disease information of the user is diabetes, the operation part 250 of the service provision server 200 may retrieve diet information for a diabetic patient previously stored in the storing part 230, but exclude a menu including a food having an intolerance level of the user in the retrieved diet information, which is equal to or higher than a predetermined reference level (for example, level 4), from the corresponding diet information, thereby generating the customized diet information for the user.

Furthermore, the operation part 250 of the service provision server 200 may retrieve a menu including only a food having an intolerance level of the user, which is less than a predetermined reference level (for example, level 4), as an ingredient through the storing part 230, and generate diet information including the retrieved food.

Moreover, the operation part 250 of the service provision server 200 may generate recommended nutritional supplement information for the user through a machine-learning analysis by using a learning model on the basis of the predicted disease information of the user and/or intolerance information of the user for each food (S340).

For example, when the predicted disease information of the user is diabetes, the operation part 250 of the service provision server 200 may retrieve nutritional supplement information for a diabetic patient previously stored in the storing part 230, thereby generating recommended nutritional supplement information for the user.

Besides, in generating customized diet information for the user whose the predicted disease information is diabetes in S330 described above, when the operation part 250 of the service provision server 200 generates customized diet information for the user by excluding a menu including a food having an intolerance level of the user, which is equal to or higher than a predetermined reference level (for example, level 4) in diet information for a diabetic patient previously stored in the storing part 230, from the corresponding diet information, the operation part 250 of the service provision server 200 may retrieve nutritional supplement containing nutrients contained in a food having an intolerance level, which is equal to or higher than a predetermined reference level (for example, level 4), through the storing part 230, thereby generating recommended nutritional supplement information capable of complementing a diet of the user.

In addition, the operation part 250 of the service provision server 200 may retrieve nutritional supplement information containing nutrients contained in a food having an intolerance level of the user, which is equal to or higher than a predetermined reference level (for example, level 4), through the storing part 230, and process the retrieved nutritional information as recommended nutritional information for the user, so that nutritional compensation for a food which may not be ingested by the user due to a risk of delayed allergy may be through recommended nutritional supplements.

The service provision server 200 may transmit the generated customized diet information and recommended nutritional supplement information for the user to the user terminal 100, so that the user may be asked to ingest a diet and nutritional supplement recommended for the user through the service according to the present invention (S350).

Meanwhile, as information on the diet and nutritional supplement, which are then ingested by the user in daily life, is recorded in the user terminal 100, the service provision server 200 may receive such recorded information from the user terminal 100 (S360).

Accordingly, the service provision server 200 may determine whether or not the ingested diet and nutritional supplement record information of the user, which has received from the user terminal 100, matches the customized diet and recommended nutritional supplement information transmitted to the user terminal 100 in S350 described above. If there is no match, the service provision server 200 may generate a message for encouraging and guiding the user to ingest the customized diet and recommended nutritional supplement (S370) and transmit the message to the user terminal 100 (S375).

Meanwhile, thereafter, as the user who continuously ingests the customized diet and recommended nutritional supplement inputs his or her own health state information into the user terminal 100, the service provision server 200 may receive the user's health state information including the expression of symptoms appearing in various diseases such as diabetes, etc.,/the amelioration of symptoms/the aggravation of symptoms, etc., from the user terminal 100 (S380), and the operation part 250 of the service provision server 200 may evaluate the property of the customized diet and recommended nutritional supplement for the user on the basis of the received health state information (S385).

Specifically, when the health condition information received from the user terminal 100 does not include symptoms related to the predicted disease information (e.g., diabetes) generated in S320 described above, the operation part 250 of the service provision server 200 may determine that the corresponding disease or a risk of occurrence thereof has been removed as the user ingests the customized diet and recommended nutritional supplement, and determine that the customized diet and recommended nutritional supplement for the user are appropriate.

In this case, the operation part 250 of the service provision server 200 may update and generate the customized diet information and the recommended nutritional supplement information for the corresponding user. However, in generating the customized diet information and the recommended nutritional supplement information as in S330 and S340 described above, the operation part 250 of the service provision server 200 may generate the customized diet information and the recommended nutritional supplement information only on the basis of the questionnaire answer information of the user for questionnaire items and the intolerance information of the user for each food without considering the corresponding predicted disease information, so that the customized diet information may be generated and the recommended nutritional supplement information may be generated through a machine learning analysis using a learning model by reflecting the user's changed health condition (S390).

As such, the service provision server 200 may transmit the updated and generated customized diet information and recommended nutritional supplement information to the user terminal 100, so that the user may receive the updated and generated customized diet information and recommended nutritional supplement information through a service according to the present invention by reflecting his/her changed health condition (S390).

In addition, in implementing the present invention, a program for executing a method for providing a machine-learning-based diet recommendation service by using a food intolerance test according to the present invention may be installed in the service provision server 200 according to the present invention or recorded in various computer-readable recording media or stored in a server for transmitting the corresponding program through a network.

Furthermore, the program according to the present invention may be distributed to computer systems connected through the network, and computer-readable codes may be stored and executed in a distributed manner, and functional programs, codes, and code segments for implementing the present invention may be easily understood by those skilled in the art to which the present invention pertains.

Meanwhile, the order of the above-described S300 to S395 in the present invention may be only an example, and is not limited thereto. In other words, the order of the above-described S300 to S395 may be changed, and some of the steps may be executed or deleted at the same time.

Terms used in the present invention are used only to describe a certain exemplary embodiment and are not intended to limit the present invention. The terms of a singular form may include plural forms unless otherwise specified. In the present application, the terms “comprise,” “have,” or the like are intended to designate that the features, the numbers, the steps, the operations, the components, the parts or combinations thereof are present, and are not to be understood as excluding the possibility that one or more other features, numbers, steps, operations, components, parts or combinations thereof may be present or added.

Although the preferred embodiments and application examples of the present invention have been illustrated and described above, the present invention is not limited to the specific embodiments and application examples described above, and various modifications may be made by those skilled in the art without departing from the gist of the present invention claimed in the claims, and such modifications should not be individually understood from the technical spirit or prospect of the present invention.

### INDUSTRIAL APPLICABILITY

The present invention may be regarded to have industrial applicability in an industrial field related to a machine-learning-based diet recommendation service.

What is claimed is:

1. A method for providing a machine-learning-based diet recommendation service by using a food intolerance test, the method comprising:
(a) generating, by a service provision server, food-specific-intolerance information of a user on a basis of a reaction value of an antigen-antibody reaction for each food antigen in a blood of the user; and
(b) generating, by the service provision server, customized diet information and recommended nutritional supplement information for the user on a basis of the food-specific-intolerance information of the user and a questionnaire answer information of the user.

2. The method of claim 1, further comprising:
after above (a) and before above (b),
generating, by the service provision server, a predicted disease information of the user on a basis of the food-specific-intolerance information of the user and the questionnaire answer information of the user.

3. A service provision server comprising:
an operation part configured to generate food-specific-intolerance information of a user on a basis of a reaction value of an antigen-antibody reaction for each food antigen in a blood of the user, and generate customized diet information and recommended nutritional supplement information for the user on a basis of the food-specific-intolerance information of the user and a questionnaire answer information of the user.

4. The service provision server of claim 3, wherein the operation part generates a predicted disease information of the user on a basis of the food-specific-intolerance information of the user and the questionnaire answer information of the user.
